(19) United States
(12) Patent Application Publication
(10) Pub. No.: US 2010/0010098 A1
(43) Pub. Date: Jan. 14, 2010

(54) POLYMORPHS OF RASAGILINE HYDROCHLORIDE

(76) Inventor: Walter Elffrink, Arnhem (NL)

Correspondence Address: SYNTHON IP INC, 7130 HERITAGE VILLAGE PLAZA, STE 202, GAINESVILLE, VA, 20155, US

(21) Appl. No.: 12/501,314

(22) Filed: Jul. 10, 2009

Related U.S. Application Data
(60) Provisional application No. 61/080,101, filed on Jul. 11, 2008.

(51) Int. Cl. A61K 31/136; C07C 211/00

(52) U.S. Cl. 514/657; 564/336

(57) ABSTRACT

A polymorph of rasagiline hydrochloride displays improved stability and is useful in forming pharmaceutical compositions for treating Parkinson's disease. Another polymorph is useful as an intermediate in forming the stable crystalline form.

This application claims the benefit of priority under 35 U.S.C. § 119(e) from prior U.S. Provisional Application Ser. No. 61/080,101, filed Jul. 11, 2008, the entire contents of which are incorporated herein by reference.

### BACKGROUND OF THE INVENTION

Rasagiline, (R)—N-2-Propynyl-1-indanamine of the formula (I)

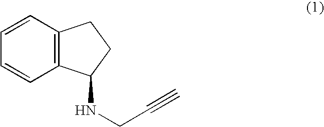

is a potent, selective, irreversible monoamine oxidase-type B (MAO-B) inhibitor for the treatment of Parkinson's disease, Alzheimer disease and various types of dementia. The compound was disclosed in EP 436-492.

In the marketed pharmaceutical formulations, the compound (1) is present as the methane sulfonate salt (i.e., rasagiline mesylate). The marketed tablet, sold under the brand name AZILECT® (Teva Pharmaceutical Industries), comprises 0.5 or 1 mg of the rasagiline mesylate and excipients of mannitol, starch, pregelatinized starch, colloidal silica, stearic acid and talc.

The original salt form of rasagiline, which was studied in the pharmaceutical and toxicological tests, was rasagiline hydrochloride. Rasagiline hydrochloride was also disclosed in EP 436492. Subsequently some stability problems were reported for rasagiline hydrochloride (see, e.g., discussion in WO 2006/057912 and particularly Example 32 of EP 812190) and a switch was made to the mesylate salt (disclosed in EP 812190).

As rasagiline hydrochloride is still an important pharmaceutical, any improvement in the state of the art relevant to this salt, particularly with the aim to solve the reported stability problems, is desirable.

### SUMMARY OF THE INVENTION

The present invention is based on the discovery of polymorphs of rasagiline hydrochloride and in particular a stable crystalline form. Accordingly, a first aspect of the present invention relates to a crystalline rasagiline hydrochloride that exhibits an x-ray powder diffraction (XRPD) pattern that includes peaks at 2θ of 8.9°, 12.2°, and 17.2°+/−0.2° at a wavelength of Cu Kα₁, (1.54056 Angstrom). Typically the XRPD pattern includes peaks at angles of about 8.9°, 12.2°, 17.2°, 21.1°, 26.2°, and 27.9°+/−0.2°. This crystalline material is often referred to herein as Form II. In some embodiments the crystalline material exhibits an XRPD that substantially corresponds to FIG. 2 herein. A Form II material that is essentially free from other forms of rasagiline hydrochloride is a particular aspect of the present invention.

Another aspect of the present invention relates to a process for making the solid state Form II of rasagiline hydrochloride, which comprises contacting a solid rasagiline hydrochloride with an inert organic liquid medium for a sufficient time to achieve conversion. In an alternative process, the Form II is formed by a crystallization of rasagiline hydrochloride Form II from a solution containing rasagiline hydrochloride dissolved therein, in the presence of seed crystals of the Form II.

In a general aspect, the invention provides a process for stabilization of rasagiline hydrochloride in a solid state, comprising converting rasagiline into rasagiline hydrochloride Form II.

A further aspect of the invention relates to yet another crystalline form designated the Form III of rasagiline hydrochloride. The Form III represents a valuable intermediate in making Form II of rasagiline hydrochloride, and thus forms a specific aspect of the present invention.

Another aspect of the invention relates to the use of the Form II in making a medicament and to pharmaceutical compositions comprising a therapeutically effective amount of the Form II of rasagiline hydrochloride, in an admixture with one or more pharmaceutically acceptable excipient(s). The composition is typically formulated for oral administration. The composition may also comprise a therapeutically effective amount of levodopa and, optionally, a therapeutically effective amount of carbidopa or a dopamine agonist.

### BRIEF DESCRIPTION OF THE DRAWINGS

FIG. 1 represents an XRPD pattern of the Form I rasagiline hydrochloride.

FIG. 2 represents an XRPD pattern of the Form II rasagiline hydrochloride.

FIG. 3 represents an XRPD pattern of the Form III rasagiline hydrochloride.

FIG. 4 represents an overlay of the XRPD patterns of rasagiline hydrochloride (abbreviated as “RSE.HCl”) Forms I-III.

The measurement conditions for the XRPD patterns are as follows:

| | |
|---|---|
| Apparatus | Powder Diffractometer Bruker D8 Advance |
| Setup | θ-2θ Bragg-Brentano reflection |
| Start angle (2θ): | 2.0° |
| End angle (2θ): | 35.0° |
| Scan step width: | 0.02° |
| Radiation type: | Cu |
| Radiation wavelengths: | 1.54060 Å (Kα₁), primary |
| | monochromator used |
| Exit slit: | 6.0 mm |
| Focus slit: | 0.2 mm |
| Divergence slit: | Variable (V20) |
| Detector: | Vantec, position sensitive detector, 6° detector |
| | opening |
| Sample holder: | Zero background silicium wafer with a cavity |

### DETAILED DESCRIPTION OF THE INVENTION

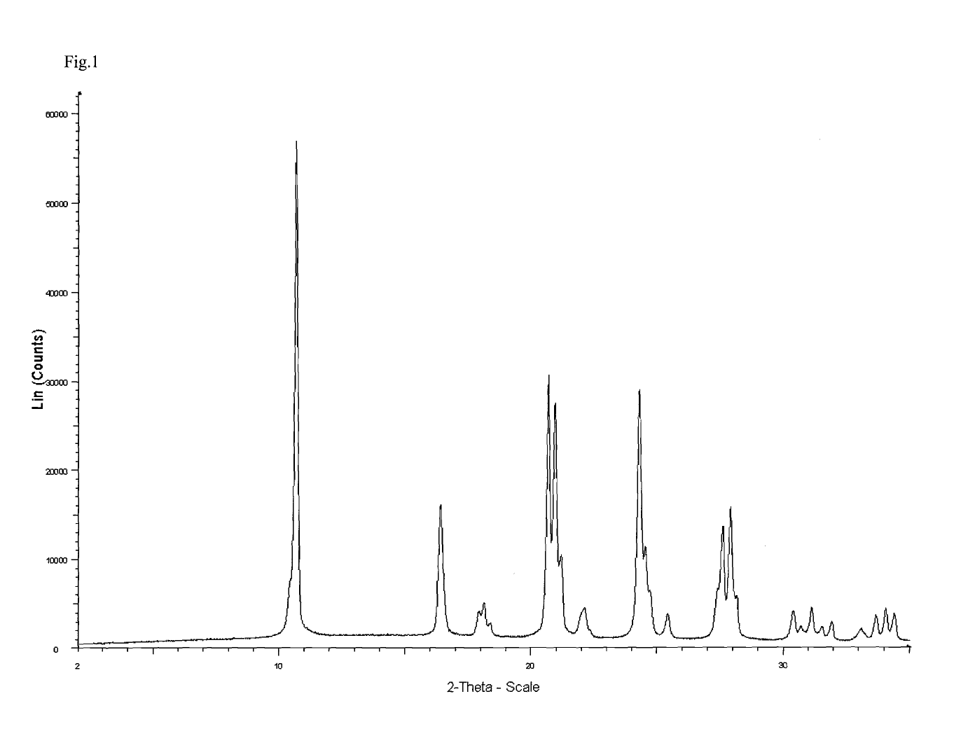

The present invention is based on the discovery of useful polymorphic forms of rasagiline hydrochloride. After repeating the prior art processes for making rasagiline hydrochloride in solid state, it was found that these processes yielded a certain crystalline form of rasagiline hydrochloride, herein denoted as the Form I. The crystalline rasagiline hydrochloride Form I can generally be characterized by an XRPD pattern as shown in FIG. 1, and has characteristic diffraction peaks at the following angles: 10.6°, 16.4°, and 24.3° 2θ+/−0.2, when measured under the above conditions. For clarity, whenever the expression “+/−0.2” is used herein it refers to the variance for each peak angle listed.

In the present invention, a crystalline polymorph of rasagiline hydrochloride was discovered. This polymorph has a different crystal structure from the Form I. This polymorph is characterized by a distinct x-ray powder diffraction (XRPD) pattern, comprising, if measured at the above conditions, signals at diffraction angles of about 21.1°, 26.2°, and 27.9°+/−0.2° 2θ. In particular, the significant or most pronounced signals appear at 8.9°, 12.2° and 17.2°+/−0.2° 2θ, while the XRPD pattern of the Form I exhibits the significant or most pronounced signals at 10.6, 16.4 and 24.3+/−0.2° 2θ.

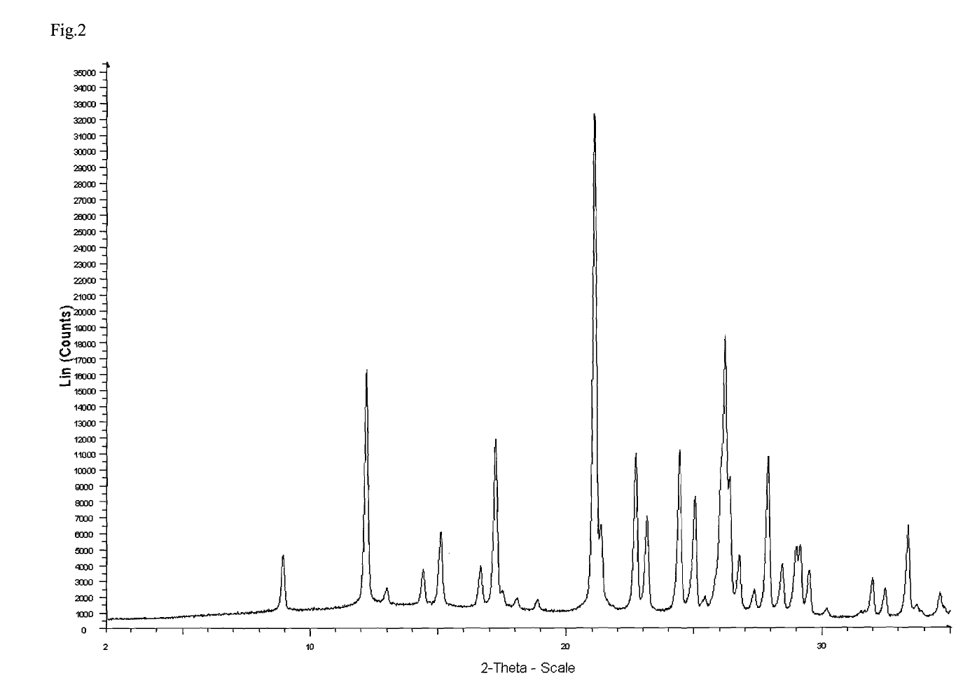

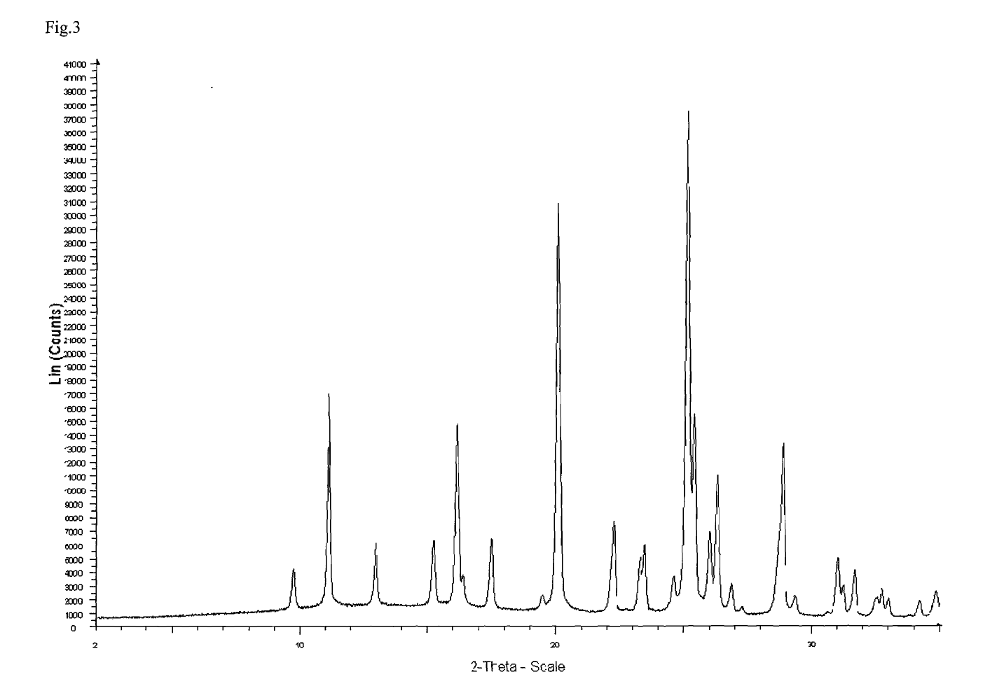

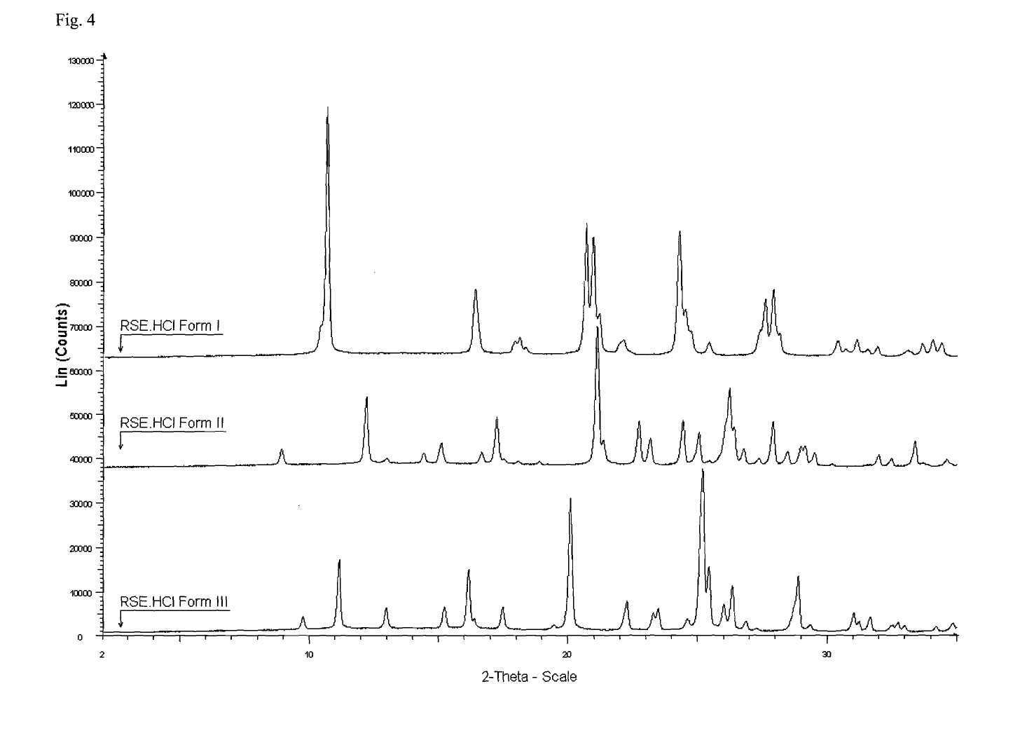

Typically, the XRPD pattern of the new polymorph substantially corresponds to that as shown in FIG. 2. The phrase “substantially corresponds” encompasses variations caused by different sample preparations, different equipment and/or settings used in measuring, normal experimental error/variation, and small amounts of impurities. Differences in a pattern or spectra that are not attributable to these factors indicate that the pattern in question does not “substantially correspond” to the reference pattern. For clarity, FIG. 1 does not substantially correspond to FIG. 2 or FIG. 3 as can be clearly seen in FIG. 4. Conversely, a spectrum may “substantially correspond” to e.g., FIG. 2, even though it is not an identical/superimposable image. Further, the XRPD pattern of an actual sample of rasagiline hydrochloride may differ from the pattern as shown in respect to intensities of the respective peaks due to preferred orientation of the crystal lattices in the powder sample.

This new polymorph of rasagiline hydrochloride of the above XRPD pattern shall be denoted in this application as “Form II of rasagiline hydrochloride,” or simply “Form II.”

The present invention includes rasagiline hydrochloride Form II as an isolated species (i.e. after removal of the residual of solvents used in the method of making it), especially in an essentially pure form and/or essentially polymorphically pure form. “Essentially pure” refers to at least 70% pure, preferably at least 80% pure, more preferably at least 90% pure, and still more preferably at least 95%, including at least 98% pure, at least 99% pure, and at least 99.8% pure, with respect to the presence of rasagiline hydrochloride in a sample. “Essentially polymorphically pure” refers to at least 70% pure, preferably at least 80% pure, more preferably at least 90% pure, and still more preferably at least 95%, including at least 98% pure, at least 99% pure, and at least 99.8% pure, with respect to the presence of the Form II in a sample. Typically in order for a sample of rasagiline hydrochloride Form II to have an XRPD pattern that substantially corresponds to FIG. 2, the Form II sample must be in essentially pure form and essentially pure polymorphical form (typically at least 90% pure polymorphically).

The finding that rasagiline hydrochloride may exist in a yet more stable, but previously unknown, polymorphic form substantially improves the potential of use of rasagiline hydrochloride in pharmaceutical formulations. In particular, the previously reported disadvantage of limited stability of rasagiline hydrochloride in bulk and in pharmaceutical compositions may be minimized by using the novel Form II thereof. Indeed, under stressed conditions rasagiline hydrochloride Form II may be more stable than rasagiline mesylate. For instance by storing a sample of Form II rasagiline hydrochloride and rasagiline mesylate for 4 weeks at 55° C./90% relative humidity, the following results were obtained:

| | Form II rasagiline | rasagiline |
|---|---|---|
| | hydrochloride | mesylate |
| Chemical Purity (HPLC) | 100% | 97.1% |
| Color Change | No | Yes, brown color |
| Water uptake | No | Yes |

Accordingly the Form II of rasagiline hydrochloride offers an equivalent and potentially superior form of rasagiline for storing or making pharmaceutical compositions.

Rasagiline hydrochloride Form II may be obtained by various processes; representative examples thereof are disclosed below. It will be apparent that any such process that comprises converting rasagiline hydrochloride into the Form II of rasagiline hydrochloride represents, in general, a process for the stabilization of rasagiline hydrochloride in a solid state.

In a first general process, rasagiline hydrochloride Form II may be obtained by a liquid-mediated solid-solid transformation of another solid state form of rasagiline hydrochloride, particularly the Form I. The process comprises contacting a solid rasagiline hydrochloride with an inert organic solvent for a sufficient time to achieve the desired conversion. In practice this contacting is typically carried out by suspending solid rasagiline hydrochloride in an inert organic liquid medium and keeping it in contact with the medium (e.g. by stirring the suspension) for a prolonged time at a suitable temperature. Depending on the duration of the contacting step, a mixture may be obtained in which the relative amount of the Form II increases with an increase in the length of time of the contact and may reach a state at which the product is essentially pure Form II.

The “inert organic liquid” for making the Form II by the above process includes, but is not limited to, a lower aliphatic alcohol, an ether, and mixtures thereof. The “lower aliphatic alcohol” preferably comprises an aliphatic alcohol/polyol with 1 to 6 carbon atoms, typically methanol, ethanol, isopropanol or tert. butanol. The “ether” preferably comprises a C3-C10 aliphatic or cyclic ether/polyether, e.g. diethyl ether, methyl tert.butyl ether, di-isopropyl ether, tetrahydrofuran, 1,4-dioxan, 1,2-dimethoxyethane (methyl cellosolve), diethylene glycol dimethyl ether (diglym) and the like.

In an advantageous arrangement, a seeding crystal of the Form II may be used to facilitate the transformation. Alternatively, starting with a mixture of forms comprising the Form II can have a similar effect as adding a seeding crystal in terms of speed, efficiency and/or completeness of conversion.

The time necessary for the conversion of the other form of rasagiline hydrochloride into the Form II is not specifically limited and may be determined by analyzing the reaction mixture and stopping the conversion process after a material comprising the desired or predetermined amount, e.g. more than 95%, of Form II is obtained. The analysis may be performed by measuring the XRPD pattern of an isolated sample and evaluating the signals at the diffraction angles that are typical for the respective forms.

The temperature of the conversion may vary from 0 to 100° C. and in some aspects is limited by the boiling point of the liquid medium.

The Form II is isolated from the liquid medium by conventional means, e.g., by filtration or centrifugation, optionally after cooling the reaction mixture to an ambient or lower than ambient temperature. The rest of the liquid medium may be removed by optional washing and drying, preferably at diminished pressure. The dried product may be further milled and/or sieved.

The starting solid rasagiline hydrochloride is not particularly limited and includes crystalline forms such as Form I, Form III, mixed crystalline materials, e.g., a substance having both Forms I and II, and amorphous forms. The starting rasagiline hydrochloride may be prepared by various processes, several of which are described below.

Isolated rasagiline hydrochloride Form I may be prepared, for instance:

a) by contacting a solution of conventional rasagiline base in diethyl ether with gaseous HCl and filtration of the precipitated product from the reaction mixture—see Example 2 or 3 of EP 436492;

b) by contacting a solution of conventional rasagiline base in isopropanol with an isopropanolic solution of HCl and filtration of the precipitated product from the reaction mixture (Example 31 of EP 812190); or

c) by recrystallization of rasagiline hydrochloride obtained sub a) from isopropanol (example 4 and 5, respectively, of EP 436492, WO 95/11016 and WO 96/37199).

The “conventional rasagiline base” comprises a product resulting from any conventional process of making rasagiline base such as:

i) a product of a chemical synthesis, e.g. of condensation of (R)-1-aminoindane with propargyl halide, advantageously pre-purified in respect to the content of side products (see, e.g., WO 2007/061717 for an example of such purification process);

ii) a product resulting from a process of optical resolution of racemic N-propargylaminoindane (prepared, e.g., as in GB 1003676 and GB 1037014); and

iii) a product obtained after treatment of a rasagiline acid addition salt with an alkali.

An example of such conventional rasagiline base is the oily material from example 2 of EP 436492, or from example 17 of WO 2007/061717, but is not limited thereto. The “conventional rasagiline base” desirably has a chemical purity of at least 95% and preferably at least 98% of chemical purity.

The Form I of rasagiline hydrochloride is characterized by the above XRPD pattern. Among the XRPD signals, any of the signals at 10.6°, 16.4°, and 24.3°+/−0.2° 2θ may be used as a characteristic signal for a determination of whether the conversion of the Form I into Form II was completed or not.

The starting rasagiline hydrochloride may also be prepared in a non-isolated state by precipitation of solid rasagiline hydrochloride after treatment of rasagiline base or other salt of rasagiline with HCl in a precipitation liquid. The “precipitation liquid” may be an organic solvent selected from a C4-C10 hydrocarbon (e.g. cyclohexane, n-hexane, benzene, toluene etc.), C3-C8 aliphatic ketone (acetone, methylethyl ketone etc.), C1-C6 aliphatic alcohol (methanol, ethanol, isopropanol, n-butanol), C4-C10 aliphatic ether (diethyl ether, methyl tert. butyl ether etc.), a cyclic ether (tetrahydrofurane, dioxan etc.), C3-C10 aliphatic ester (ethyl acetate etc.), a nitrile (acetonitrile) and mixtures thereof.

In an advantageous mode, the so formed suspension of rasagiline hydrochloride is then allowed to stay in contact with that precipitation liquid to achieve the conversion of the precipitated rasagiline hydrochloride into the Form II as shown above, i.e. the precipitation liquid also serves as the “inert organic liquid” for the liquid-mediated solid-solid conversion. Alternatively, the precipitation liquid may be, at least partly, removed and/or replaced by the inert organic liquid as defined above.

In a special aspect, a previously undisclosed Form III of rasagiline hydrochloride may be prepared and used as the starting rasagiline hydrochloride for the liquid-mediated solid-solid conversion. A characteristic XRPD pattern of Form III is depicted in FIG. 3. Among the signals in the Form II XRPD, the signal at 8.9°, 12.2°, 17.2°, and/or 21.1°+/−0.2° 2θ may be used as a characteristic signal to determine whether the conversion of the Form III into Form II has occurred.

The Form III may be prepared by addition of hydrochloric acid to a solution of rasagiline in 2-propanol at a temperature close to the reflux temperature and precipitation of the rasagiline hydrochloride at this temperature.

Similarly as disclosed above for the Form II, the present invention includes rasagiline hydrochloride Form III as an isolated substance, especially in an essentially pure form. The present invention also includes mixtures of the Form III with other forms of rasagiline hydrochloride, especially with the Form I and/or with the Form II.

In a second general process, the Form II may be prepared by a crystallization of rasagiline hydrochloride from a solution thereof, wherein the crystallization is carried out in the presence of a seed crystal of the Form II. In such process, the rasagiline hydrochloride starting material may be:

i) the known Form I, the newly discovered Form III or any other solid state form of rasagiline hydrochloride as well as mixtures thereof;

ii) non-solid forms of rasagiline hydrochloride (such as oils or elaborated reaction mixtures); or

iii) in-situ formed rasagiline hydrochloride (by contacting rasagiline base with HCl directly in the crystallization solvent).

The crystallization is generally performed by providing a solution of the rasagiline hydrochloride in a solvent (i.e., a “crystallization solvent”), optionally by heating to the reflux temperature, adding the seed crystal of the Form II and precipitating the Form II therefrom. The precipitation of the crystals (i.e. “crystallization”) may be further aided or induced by cooling the solution. If necessary or if desirable, the solution may also be pre-treated by a surface active material, e.g., by activated carbon, celite or silica gel, to remove matters that would negatively affect the ability to crystallize or that would decrease the overall purity. This material is advantageously removed by filtration at enhanced temperature before the precipitation. The cooling is often performed under stirring. The seeding crystal of the Form II should be placed into the solution at a proper temperature; namely a temperature at or near the temperature corresponding to a saturated concentration of rasagiline hydrochloride in the crystallization solvent.

The crystallization solvent advantageously consists of, or at least contains, an alcohol, preferably C1-C4 aliphatic alcohol, and most preferably 2-propanol. Other solvents such as those mentioned above as precipitation liquids may also be present. For example, in forming rasagiline hydrochloride in situ, the rasagiline base may be provided as a solution in a C4-C10 hydrocarbon (aliphatic or aromatic) such as toluene, which is then contacted with a C1-C4 alcoholic solution of hydrogen chloride. The resulting crystallization solvent thus contains the advantageous C1-C4 alcohol and the toluene co-solvent.

The isolation of the formed solid (by filtration, washing) is advantageously performed at ambient or lower than ambient temperatures. Advantageously, the suspension of the crystallized product before isolation is stirred for a certain time (e.g. from 30 minutes to several hours), to assure that any unstable solid state forms (e.g. Form I or Form III), whenever formed, are converted into the stable Form II. Drying is advantageously performed in vacuo, preferably at a temperature not exceeding 25° C.

The nature of the crystallization solvent, concentration, cooling rate and the final temperature may affect the shape of the formed crystals and yield. It is within a routine experimentation to adjust proper crystallization regimen. The precipitated crystals preferably are not formed in a long needle shape, as such form is less useful in pharmaceutical applications, as known in the art. Therefore, the formed crystals should preferably have the aspect ratio less than 15, preferably less than 10. As used herein, “aspect ratio” is the quotient of the division of a crystal's length by its width. The aspect ratio can be obtained by taking micrographs of a batch of crystals and measuring the length and width of a plurality of representative crystals.

Furthermore, it is preferred that the formed crystals have an average particle size of less than 1000 microns, preferably less than 250 microns and more preferably less than 100 microns. The product of small particle size is, in general, easier to process in forming pharmaceutical compositions than a large particle size. A relatively quick cooling rate, high speed of stirring, and adding the seed crystal at the proper temperature may aid in making the product with sufficiently small particle size and with the desired aspect ratio. Otherwise conventional operations like sieving or milling can be performed to achieve a desired particle size.

The chemical purity of the starting rasagiline hydrochloride (the content of rasagiline in respect to the content to all structurally related compounds) should be preferably at least 95%, otherwise the impurities present can decrease the yield of the precipitated product. Therefore, the solution comprising the rasagiline hydrochloride can be pre-purified before precipitation, e.g. by filtration with an activated carbon, by an extraction, by chromatography, etc., if desired.

On the other hand, the crystallization process according to the present invention itself serves as a useful purification tool; e.g., the crude 95% rasagiline hydrochloride product can be converted to pure 99% rasagiline hydrochloride Form II by a single or repeated crystallization. Preferably, the rasagiline hydrochloride Form II of a chemical purity higher than 98% may be produced without employing any other subsequent purification process. The crystallization process may also enhance the enantiomeric purity of the product. For example rasagiline hydrochloride Form II of higher than 98% enantiomeric purity may be made this way.

It is apparent from the above that the Form II is a more stable form of rasagiline hydrochloride, at least in a suspension with a liquid. This represents an advantage, e.g. in a wet granulation processes for making tablets or in pelletization processes. The solid state stability of the Form II under contact with granulation or pelletization liquid minimizes the danger of a polymorphic change of rasagiline hydrochloride during the process, which could cause changes in properties of the resulting granulate or pellet. In particular, the Form II is more stable in the solid state than the crystalline Form I obtainable by the prior art procedures.

Rasagiline hydrochloride Form II can be formulated into various pharmaceutical compositions with one or more pharmaceutically acceptable excipients. The pharmaceutical composition can be in a unit dosage form, such as a solid oral dosage form (i.e. tablet or capsule), a solution or suspension (especially for an aqueous sterile solution or suspension for parenteral administration), or bulk precursors thereof, such as a pre-blended mixture ready for further blending/addition of ingredients or a blend ready for tabletting or filling into capsules. Making a pharmaceutical composition from rasagiline hydrochloride Form II; e.g., made by a process which comprises combining crystalline rasagiline hydrochloride Form II with said at least one pharmaceutically acceptable excipient, as well as the resulting pharmaceutical composition are specific aspects of the present invention. Further the invention includes pharmaceutical compositions comprising a therapeutically effective amount of the Form II of rasagiline hydrochloride, in an admixture with one or more pharmaceutically acceptable excipient(s).

Pharmaceutically acceptable excipients are known in the art and include carriers, diluents, fillers, binders, lubricants, disintegrants, glidants, colorants, pigments, taste masking agents, sweeteners, flavorants, plasticizers, and any acceptable auxiliary substances such as absorption enhancers, penetration enhancers, surfactants, co-surfactants, and specialized oils. The proper excipient(s) are selected based in part on the dosage form, the intended mode of administration, the intended release rate, and manufacturing reliability. Examples of common types of excipients include various polymers, waxes, calcium phosphates, sugars, etc. Polymers include cellulose and cellulose derivatives such as HPMC, hydroxypropyl cellulose, hydroxyethyl cellulose, microcrystalline cellulose, carboxymethylcellulose, sodium carboxymethylcellulose, calcium carboxymethylcellulose, and ethylcellulose; polyvinylpyrrolidones; polyethylenoxides; polyalkylene glycols such as polyethylene glycol and polypropylene glycol; and polyacrylic acids including their copolymers and crosslinked polymers thereof, e.g., Carbopol® (B.F. Goodrich), Eudragit® (Rohm), polycarbophil, and chitosan polymers. Waxes include white beeswax, microcrystalline wax, carnauba wax, hydrogenated castor oil, glyceryl behenate, glycerylpalmito stearate, and saturated polyglycolyzed glycerate. Calcium phosphates include dibasic calcium phosphate, anhydrous dibasic calcium phosphate, and tribasic calcium phosphate. Sugars include simple sugars, such as lactose, maltose, mannitol, fructose, sorbitol, saccharose, xylitol, isomaltose, and glucose, as well as complex sugars (polysaccharides), such as maltodextrin, amylodextrin, starches, and modified starches. In some embodiments, a polymer that is able to form a molecular dispersion with rasagiline hydrochloride is used as an excipient. An example of such a polymer is polyvinylpyrrolidone hydroxypropylmethylcellulose phthalate. Such a dispersion can be formed by methods well known in the art, for example, dissolving the rasagiline hydrochloride and the polymer in a suitable solvent and evaporating the solvent.

The Form II rasagiline hydrochloride may be preferably formulated into compositions for oral administration. The compositions for oral administration may be solid or liquid, such as in the form of an oral solution, oral capsule, or an oral tablet, and may exhibit immediate release or, preferably, modified and/or extended release of the active substance from the composition. The pharmaceutical compositions comprising Form II rasagiline hydrochloride may be formulated, for instance, into conventional oral tablets. The tablets may be monolithic tablets, i.e. tablets that upon ingestion do not disintegrate into a plurality of smaller units from which the active ingredient is finally released, or may be disintegrable tablets. The tablets may be produced by any standard tabletting technique, e.g. by wet granulation, dry granulation, or direct compression.

The wet granulation procedure comprises mixing rasagiline hydrochloride and excipients with a granulation liquid and forming a solid granulated mass, which is then optionally dried, screening the granulate, mixing with remaining excipients, in particular with a lubricant, and compressing into a tablet.

The dry granulation procedure typically comprises mixing the solid excipients (except lubricants), compacting the mixture in a compactor (e.g., a roller compactor), milling the compacted mass, screening the milled granules, mixing with a lubricant, and compressing the mixture into tablets.

The direct compression procedure generally comprises mixing the solid excipients and compressing the uniform mixture into tablets.

Rasagiline hydrochloride Form II may also be formulated by melt granulation, i.e., in an admixture with a functional excipient (e.g., glyceryl behenate) that melts at elevated temperature and forms a granulateable melt that is granulated in suitable equipment.

Further, chewable or oral-dispersible tablets are also contemplated as oral tablets.

As an example of suitable pharmaceutical tablets, the rasagiline hydrochloride Form II may be blended with excipients that are used in the marketed AZILECT® tablets (mannitol, pregelatinized starch, maize starch, talc, stearic acid, and colloidal silica) to produce tablets comprising the same amount of rasagiline (in respect to content of the free base).

Rasagiline hydrochloride Form II may also be blended into compositions that are suitable for being formulated into pellets by known pelletization techniques. A plurality of rasagiline pellets comprising a single dose of rasagiline base may be encapsulated into capsules made from pharmaceutically acceptable material, such as hard gelatin. In another mode, a plurality of pellets may be compressed together with suitable binders and disintegrants to a disintegrable tablet that, upon ingestion, decomposes and releases the pellets. In yet another mode, the plurality of pellets may be filled into a sachet.

The relative amount of the rasagiline hydrochloride Form II in the tablet or pellet mass is not particularly limited but typically may range from 1 to 10 wt %, such as 2 to 5 wt %.

Immediate release solid oral compositions comprising rasagiline typically have the following release profile: more than 80% of the active is released in 30 minutes, preferably in 15 minutes, when measured by the paddle method of Ph.Eur at 50 rpm in 0.01 M HCl. Extended release solid oral compositions have, advantageously, a release profile in which less than 60% rasagiline is released from the composition in 3 hours, when measured by the paddle method of Ph.Eur at 50 rpm in 0.01 M HCl.

Tablets or pellets may be coated by a suitable film coat, which may be a film coat (dissolvable in the stomach) or an “enteric coat” (not dissolvable in the stomach). The film coat may protect the tablet against the environment (light, air, moisture) during storage and handling. Any conventional film coat may be used. The enteric coat retards or inhibits the release of rasagiline in the stomach.

In a particular embodiment, the pharmaceutical composition is substantially free of the rasagiline hydrochloride Form I, i.e. the pharmaceutical composition contains less than 2%, more preferably less than 1%, of Form I relative to the sum of all forms therein.

The unit dose in tablet form may comprise a single tablet but it may also comprise a divided tablet or several smaller tablets (minitablets) administered at the same time. The unit dose of pellets in capsule form may comprise a single capsule. Solutions for oral administration may be packed in a multidose package, the unit dose being packaged in a calibrated vessel.

The pharmaceutical dosage forms formulated from the compositions of the invention may comprise a unit dose of rasagiline, i.e., a therapeutically effective amount of rasagiline hydrochloride for a single dose administration. The amount of the Form II rasagiline hydrochloride, expressed as rasagiline base, in the daily unit dose may range from 0.01 to 10 mg, typically from 0.2 to 2 mg.

In addition to the rasagiline hydrochloride Form II, the pharmaceutical compositions of the present invention can also contain a therapeutically effective amount of other active ingredient(s). Most importantly, such active ingredients may be compounds that are co-active in treatment of the diseases treatable by rasagiline, particularly Parkinson's disease. For example levodopa, alone or in combination with other active ingredients that enable more levodopa to enter the brain before it converts to dopamine, such as carbidopa or benserazide, can be used in combination with the rasagiline hydrochloride composition. The combination can be in the same dosage form, i.e., both actives in a single tablet, or as separate dosage forms. Besides levodopa, or in addition thereto, other dopamine agonists such as pergolide, lisuride, and pramipexole may be used in combination with rasagiline hydrochloride compositions.

The pharmaceutical compositions comprising solid rasagiline hydrochloride Form II of the present invention are useful in preventing, ameliorating or treating Parkinson's disease and various other conditions, such as depressions, Attention Deficit Disorder (ADD), Attention Deficit and Hyperactivity Disorder (ADHD), Tourett's syndrome, Alzheimer's disease and other dementias. They may be used in monotherapy or in a combined therapy, e.g. with levodopa.

The present invention is more particularly described and explained by the following non-limiting examples.

### EXAMPLES

### Example 1—Comparative

### Form I

### Similar to WO 96/37199 Example 3

1.08 g of rasagiline free base was dissolved in 10 ml of diethyl ether. The flask was placed in an ice bath (no stirring). HCl (g) was bubbled through the solution (˜1 minute). After termination a sample of the solid was isolated by filtration and analysed by XRPD: rasagiline hydrochloride Form I.

### Example 2—Comparative

### Form I

### Procedure of Ex 31 of EP 812190

0.50 g rasagiline free base was dissolved in 5 ml 2-PrOH at RT. 0.62 ml 5 M HCl in 2-PrOH was added in one minute. A white suspension was formed. After 15 minutes a sample of the solid was taken and analysed by XRPD: Form I.

### Example 3—Comparative

### Form I

### Crystallization

5.09 g rasagiline free base was dissolved in 22 ml hot 2-PrOH. 6.00 ml 5 M HCl in 2-PrOH was added in one minute. After about one minute solid material crystallized. A sample was taken after 15 minutes of stirring. The isolated solid was analysed by XRPD: Form I. The mixture was stirred at RT for 2 hours at 5° C. and was isolated by filtration.

XRPD: Form I.

### Example 4

### Form II from Form I

9 mg of rasagiline HCl Form I was suspended in 0.15 ml 2-PrOH and stirred for 11 days at 70° C.

Form II was obtained.

b) 50 mg of rasagiline HCl Form I was suspended in 0.23 ml 2-PrOH and stirred for 7 days at 5° C.

Form II was isolated.

### Example 5

### Form II from Form III

20 mg of Form II was added to a suspension of 1.49 g Form III in 6 ml 2-PrOH. After 4 days solid material was isolated by filtration, washed with diethyl ether and was dried overnight at 40° C. in vacuo.

XRPD: Form II.

### Example 6

2.55 g rasagiline free base was dissolved in 10 ml 2-PrOH. 3.0 ml ˜5 M HCl in 2-PrOH was added in one minute. A suspension was formed. Immediately ˜30 mg of seeds of Form II were added. After 5 minutes of stirring a sample was isolated and analysed by XRPD: Form II.

After one hour of stirring solid material was isolated by filtration, washed with Et₂O, dried overnight at 40° C. in vacuo. Yield: 1.62 g. XRPD: Form II

### Example 7

### Preparation of Form II

27.76 g of toluene solution of rasagiline base comprising 8.32 g of rasagiline was heated to 75° C. and, while stirring, 15 ml of 15% HCl in 2-PrOH was added in one minute. The solution was seeded at 80° C. with seeds of rasagiline HCl Form II, the reaction mixture was cooled to 65° C. during 10 minutes and stirred for 30 minutes at 65° C. The suspension was cooled to 25° C. during 1 h. Stirred for 14 h, filtered and washed with 10 ml of 2-PrOH. Dried to constant mass. Yield: 8.160 g. XRPD: Form II

### Example 8

1.08 g rasagiline HCl Form III was dissolved in 10 ml hot 2-PrOH. 20 mg of seeds of rasagiline HCl Form II were added. The mixture was stirred for 3 hours, was filtered, washed with Et₂O and dried overnight at 40° C. under reduced pressure.

XRPD: Form II

### Example 9

### Preparation of Form III

1.49 g rasagiline free base was dissolved in 6 ml 2-PrOH at RT. The solution was heated in a 100° C. oilbath. In about one minute 1.80 ml 5M HCl in 2-PrOH was added to the hot solution. A suspension was formed. The flask was cooled to RT and after 1.5 h of stirring solid material was isolated by filtration, washed with Et₂O and dried overnight under reduced pressure at 40° C. Yield: 1.38 g. XRPD: Form III.

Each of the patents and patent applications mentioned above are incorporated herein by reference. The invention having been described it will be obvious that the same may be varied in many ways and all such modifications are contemplated as being within the scope of the invention as defined by the following claims.

I claim:

1. A crystalline rasagiline hydrochloride that exhibits an XRPD pattern having peaks at 2θ of 8.9°, 12.2°, and 17.2°+/−0.2°.

2. The crystalline rasagiline hydrochloride according to claim 1, wherein said XRPD pattern contains peaks at 2θ of 8.9°, 12.2°, 17.2°, 21.1°, 26.2°, and 27.9°+/−0.2°.

3. The crystalline rasagiline hydrochloride according to claim 1, which exhibits an XRPD pattern that substantially corresponds to FIG. 2.

4. The crystalline rasagiline hydrochloride according to claim 1, having a particulate form and an average particle size below 250 microns.

5. The crystalline rasagiline hydrochloride according to claim 4, wherein the particles have an aspect ratio of less than 10.

6. The crystalline rasagiline hydrochloride according to claim 2, in isolated state and having a chemical purity of at least 90%.

7. The crystalline rasagiline hydrochloride according to claim 6, having a polymorphic purity of at least 90%.

8. The crystalline rasagiline hydrochloride according to claim 2, having a particulate form, an average particle size below 100 microns, and an aspect ratio of less than 15.

9. A process for making the crystalline rasagiline hydrochloride according to claim 1, which comprises contacting a solid rasagiline hydrochloride with an inert organic liquid medium for a sufficient time to achieve the conversion of said solid rasagiline hydrochloride into said crystalline rasagiline hydrochloride.

10. A process for making the solid state Form II of rasagiline hydrochloride, comprising crystallizing form II rasagiline hydrochloride from a solution containing rasagiline hydrochloride dissolved in a solvent, wherein said crystallizing step is carried out in the presence of seed crystals of the Form II.

11. The process according to claim 10, wherein said solvent comprises a C1-C4 aliphatic alcohol.

12. The process according to claim 10, which further comprises contacting a solution of rasagiline base with hydrogen hydrochloride to form said solution of rasagiline hydrochloride.

13. The process according to claim 12, wherein said solution of rasagiline base comprises toluene as the solvent and wherein a 2-propanol solution of hydrogen chloride is used to contact hydrogen chloride with said solution of rasagiline base.

14. A pharmaceutical composition comprising a therapeutically effective amount of the Form II of rasagiline hydrochloride, in an admixture with one or more pharmaceutically acceptable excipient(s).

15. The pharmaceutical composition according to the claim 14, which further comprises a therapeutically effective amount of levodopa, carbidopa, a dopamine agonist, or combinations thereof.

16. A pharmaceutical composition, comprising rasagiline hydrochloride and at least one pharmaceutically acceptable excipient, wherein said composition was made by a process which comprises combining crystalline rasagiline hydrochloride Form II with said at least one pharmaceutically acceptable excipient.

17. A crystalline form of rasagiline hydrochloride, wherein said crystalline form is Form III.
